(19) United States
(12) Patent Application Publication
(10) Pub. No.: US 2025/0325261 A1
(43) Pub. Date: Oct. 23, 2025

(54) Surgical Instrument and Methods of Tissue Repair

(71) Applicant: Arthrex, Inc., Naples, FL (US)

(72) Inventors: Shaun G. LEBLANC, Naples, FL (US); Bradford O. PARSONS, Irvington, NY (US); John SEYLER, Naples, FL (US); Stephan M. BOSCH, Naples, FL (US)

(73) Assignee: Arthrex, Inc., Naples, FL (US)

(21) Appl. No.: 19/059,434

(22) Filed: Feb. 21, 2025

Related U.S. Application Data
(60) Provisional application No. 63/634,958, filed on Apr. 17, 2024.

(51) Int. Cl. A61B 17/04

(52) U.S. Cl. CPC A61B 17/0483

(57) ABSTRACT

Instruments and methods for grasping, retrieving, and passing suture in tissue repairs and other surgical procedures are disclosed.

### BACKGROUND

The disclosure relates to the field of surgery and, more specifically, to surgical instruments for suturing tissue and associated methods of tissue repairs.

### SUMMARY

Surgical instruments and methods of tissue repairs are disclosed. A suture grasper/retriever/passer instrument can have a double jaw independently operable to retrieve two or more flexible strands and prevent tangling of the strands.

A surgical instrument can be a suture retriever designed to grasp and retain multiple sutures to aid in suture management. The instrument is provided with a handle and an end effector having three jaws movably coupled for retrieving and retaining flexible materials such as sutures. In an embodiment, two of the three jaws are movable between an open position in which the distal ends of the two jaws are spaced apart from a fixed, non-movable jaw, and a close position in which the distal ends directly contact the fixed, non-movable jaw. Each of the two movable jaws can define an opening when the jaws are in the close position. When in close position, the two movable jaws of the instrument form two separate enclosures for suture retention.

Methods of grasping/retrieving/passing suture and suturing tissue are also disclosed. In an embodiment, different flexible strands can be retained in different enclosures of a suture grasper instrument and can be passed without tangling, twisting or entwining.

### BRIEF DESCRIPTION OF THE DRAWINGS

FIG. 1 illustrates a surgical instrument.

FIG. 2 is an enlarged perspective view of the distal end of the surgical instrument of FIG. 1 (in the open position).

FIG. 3 is another view of the distal end of the surgical instrument of FIG. 1 (in the open position).

FIG. 4 is an enlarged perspective view of the distal end of the surgical instrument of FIG. 1 (in the close position).

FIG. 5 illustrates an enlarged perspective view of the distal end of the surgical instrument of FIG. 1 (in the close position) employed in tissue repair.

FIG. 6 illustrates a distal end of another surgical instrument.

FIG. 7 illustrates a distal end of another surgical instrument.

FIG. 8 illustrates another surgical instrument.

### DETAILED DESCRIPTION

The disclosure provides surgical instruments and methods for securing a first tissue to a second tissue (for example, soft tissue to another tissue) with a suture passer/grasper/retriever instrument configured to grasp and retain flexible strands at different locations on the instrument and to prevent the retained sutures from tangling during a surgical procedure, aiding therefore in suture management.

A surgical instrument can be a suture passer. A surgical instrument can be a suture grasper. A surgical instrument can be a suture retriever.

A surgical instrument can include a shaft, an actuating mechanism, and a jaw assembly. A jaw assembly can include a first jaw member, a second jaw member, and a third jaw member, the first jaw member and the second jaw member being movable with respect to each other and to the third jaw member, between a close position and an open position. In the closed position, the jaw assembly can form two distinct and separate enclosures for retaining separate flexible strands. An actuating mechanism can be coupled to the jaw assembly and configured to move the jaw assembly between the close position and the open position. A surgical instrument can also include a handle assembly.

In an embodiment, a surgical instrument comprises: (i) a shaft having a longitudinal axis, a distal end, and a proximal end; (ii) a jaw assembly at a tip of the distal end of the shaft, the jaw assembly being configured to close to form two openings to allow secure placement and holding of different flexible strands (for example, sutures, suture wires, or similar strands); and (iii) an actuator configured to move the jaw assembly. An actuator can be within the shaft and at the distal end of the shaft. A surgical instrument can be a suture grasper, suture retriever, and/or suture passer.

A jaw assembly can include two movable jaws (movable members) and a stationary jaw (stationary member). Each of the two movable jaws can be movable with respect to the stationary jaw. The two movable jaws can have different lengths or can have similar lengths. The two movable jaws can be located on same side, or on opposite sides relative to the stationary jaw.

A jaw assembly can include one movable jaw (movable member) and a stationary jaw (stationary member). The movable jaw can be movable with respect to the stationary jaw. The movable jaw and the stationary jaw can have about similar lengths and can be located on opposite sides relative to the longitudinal axis of the shaft. The movable jaw and the stationary jaw can form at least two enclosures for securely retaining a plurality of strands when in a close position.

In another embodiment, a surgical instrument comprises: (i) a shaft having a longitudinal axis, a distal end, and a proximal end; (ii) a first movable jaw having a first length and a second movable jaw having a second length, wherein the first length is different from the first length, wherein the first movable jaw and the second movable jaw are movable individually and independently with respect to each other; and (iii) a mounted stationary jaw (stationary member or tip) fixedly mounted to the shaft at the distal end of the shaft, the stationary jaw having two spaced apart and mounting locations/sites for securely engaging a most distal tip of the first movable jaw and a most distal tip of the second movable jaw. A surgical instrument can also include a handle mechanism configured to move the first movable jaw and the second movable jaw with respect to the mounted stationary jaw. A handle mechanism can include two handles with finger loops. A handle mechanism can further include a locking mechanism for locking the jaw assembly. A surgical instrument can also include an actuator. An actuator can be within the shaft and at the distal end of the shaft.

In an embodiment, a suture retriever instrument comprises: a shaft having a longitudinal axis, a distal end, and a proximal end; and a securing mechanism that is located at the distal end of the shaft and that moves from a first configuration to a second configuration when at least one flexible strand is secured therein. A securing mechanism can grasp, retrieve, and secure one or more flexible strand (e.g., suture). A securing mechanism can include at least two separate, distinct, adjacent, continuous openings or enclosures. The at least two openings/enclosures can have about similar dimensions. The at least two openings/enclosures can have different dimensions.

Methods of suture management are also disclosed. In an embodiment, suture can be grasped, retained, and secured with a surgical instrument by securing two or more flexible strands within two or more distal openings/enclosures at the tip of the instrument.

An exemplary method of securing a plurality of flexible strands to a surgical instrument comprises: (i) positioning a first flexible strand of the plurality of flexible strands within a first region of a stationary jaw at a tip of the instrument; (ii) positioning a second flexible strand of the plurality of flexible strands within a second region of the stationary jaw at the tip of the instrument; and (iii) securing the first flexible strand and the second flexible strand to the instrument. The method can further include actuating at least one movable jaw at the tip of the instrument to bring the at least one movable jaw in contact with the stationary jaw and to form two separate and distinct openings or enclosures for securing and retaining the first flexible strand and the second flexible strand. The method can further include actuating a first movable jaw at the tip of the instrument to bring the first movable jaw in contact with the stationary jaw and to form a first opening or enclosure for securing and retaining the first flexible strand within the first opening; and actuating a second movable jaw at the tip of the instrument to bring the second movable jaw in contact with the stationary jaw and to form a second opening or enclosure for securing and retaining the second flexible strand within the second opening. The method can further include removing the surgical instrument from a surgical site without tangling the first flexible strand with the second flexible strand.

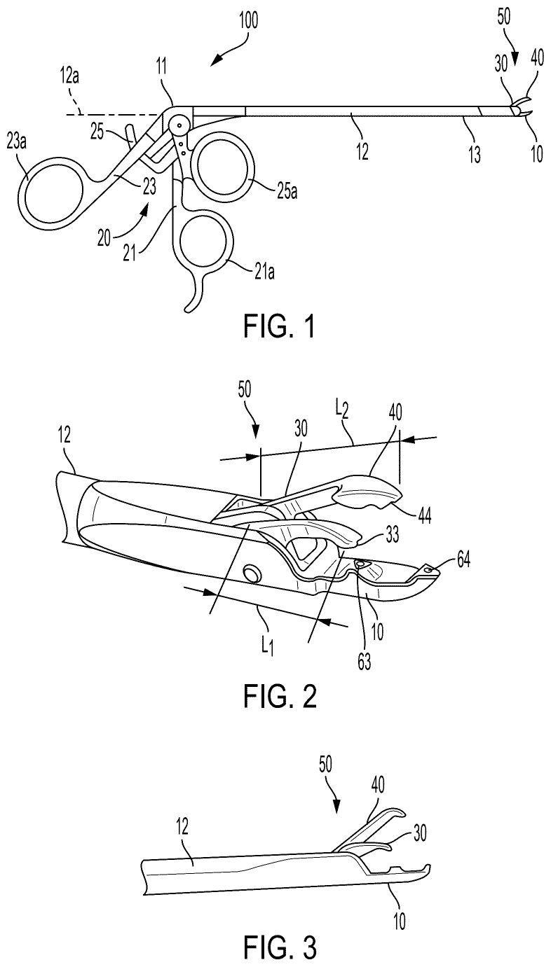

Referring now to the drawings, where like elements are designated by like reference numerals, FIGS. 1-8 illustrate structural elements of surgical instrument 100, 200, 300, 400 (device 100, 200, 300, 400; suture grasper 100, 200, 300, 400; suture retriever 100, 200, 300, 400; suture passer 100, 200, 300, 400; multiple-jaw instrument 100, 200, 300, 400; double-opening jaw instrument 100, 200, 300, 400; grasper/retriever/passer instrument 100, 200, 300, 400).

As shown in FIG. 1, instrument 100 includes an elongated tubular member 12 (shaft 12) having a longitudinal axis 12a, a proximal end 11, a distal end 13, and an axial throughbore therein (not shown). Elongated tubular member 12 may be a tube or a narrow-diameter rod of dimensions that permit the tubular member to be introduced through an associated cannula (for example, an 8.25 cannula) in a minimally invasive procedure, such as arthroscopic or other endoscopic procedures, or into a body cavity, such as the abdominal cavity.

Elongated tubular member 12 connects a handle assembly 20 with a jaw assembly 50 formed of jaws 10, 30, 40. Stationary jaw 10 (stationary member 10; static member 10; tip 10; mounted stationary jaw 10) is provided at the distal end 13 of the instrument and is preferably integral with (fixedly mounted to) the tubular member 12 at the distal end 13 of the shaft. Jaws 30, 40 (first and second movable jaws 30, 40; movable members 30, 40) are movable with respect to the stationary jaw 10 and with respect to each other.

Reference is now made to FIGS. 2 and 3 which illustrate enlarged views of the jaw assembly 50 at the distal end 13 of the tip of instrument 100. Stationary jaw 10 is fixedly mounted to the shaft 12 at the distal end of the shaft. Stationary jaw 10 is provided with at least one contacting location/site 63 (mounting location/site 63; engagement location/site 63) for securely engaging a most distal tip 33 of the first movable jaw 30 when the jaw assembly 50 is in a close position or close configuration. In an embodiment, the contacting location/site 63 is located at about the mid length of the stationary jaw 10. A most distal tip 44 of the second movable jaw 40 securely engages a most distal tip 64 of the stationary jaw 10, when the jaw assembly 50 is in a close position or close configuration. In an exemplary embodiment, stationary jaw 10 is provided with two spaced apart and different contacting locations/sites 63, 64 for securely engaging a most distal tip 33 of the first movable jaw 30 and a most distal tip 44 of the second movable jaw 40. Stationary jaw 10 is substantially straight with respect to the shaft 12.

As shown in FIG. 2, first movable jaw 30 has a first length L1 and second movable jaw 40 has a second length L2, wherein the first length L1 is different from the second length L2. In an exemplary-only embodiment, the first length L1 is smaller than the second length L2. The first movable jaw 30 and the second movable jaw 40 can be moved individually and independently with respect to each other and to the stationary jaw 10. First and second movable jaws 30, 40 can be also moved simultaneously, from an open position (first position) to a close position (second position). The first movable jaw 30 and the second movable jaw 40 can be pivotably movable jaws/members. The first movable jaw 30 and the second movable jaw 40 can be straight (as shown in FIG. 2) or generally curved. The first movable jaw 30 and the second movable jaw 40 form part of the tip of the instrument 100 and can pivot with respect to the tip or the stationary jaw 10.

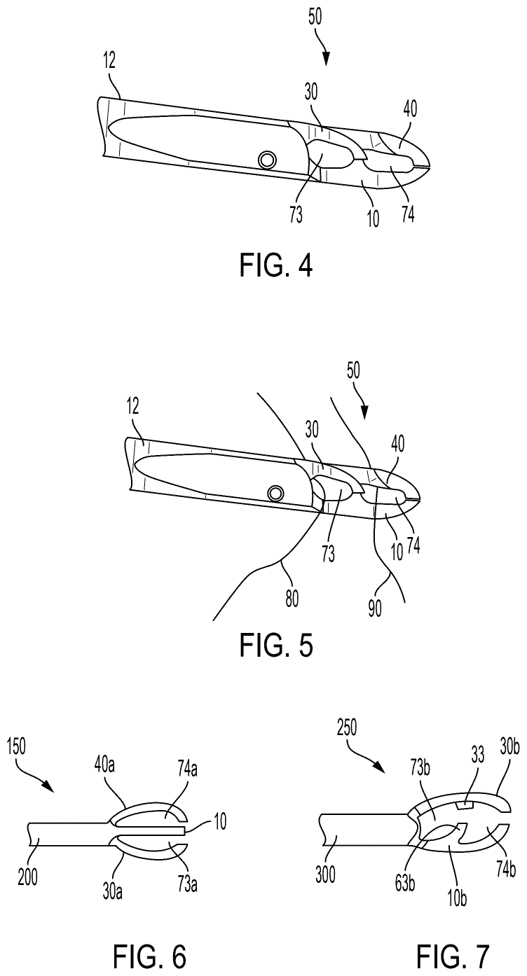

FIGS. 2 and 3 illustrate the jaw assembly 50 in a first position or an open position, i.e., first and second movable jaws 30, 40 are spaced apart from stationary jaw 10. FIG. 4 illustrates the jaw assembly 50 in a second position or a fully close position, i.e., with first and second movable jaws 30, 40 engaging the stationary jaw 10 to form a first opening 73 (first enclosure 73; first through-opening 73; first aperture 73) and a second opening 74 (second enclosure 74; second through-opening 74; second aperture 74). When in the fully close position, the most distal tip 33 of the first movable jaw 30 engages the first mounting location/site 63 of the stationary jaw, and the most distal tip 43 of the second movable jaw 40 engages the second mounting location/site 64 of the stationary jaw. First and second openings 73, 74 are about coplanar with the longitudinal axis 12a of shaft 12. First and second openings 73, 74 are separate, distinct, adjacent, and continuous openings. First and second openings 73, 74 can have similar or different dimensions and can each hold at least one flexible strand such as suture. First and second openings 73, 74 can have an oval configuration or similar configurations. First and second openings 73, 74 are formed when the first and second movable jaws 30, 40 and the stationary jaw 10 are in the fully close position.

FIG. 5 illustrates jaw assembly 50 of instrument 100 with two flexible strands 80, 90 secured to the instrument. First flexible strand 80 is positioned and secured within the first opening 73. Second flexible strand 90 is positioned and secured within the second opening 74.

Each of openings 73, 74 may include a transversal suture loading slot which can be arcuate, for example, to allow suture 80, 90 or a strand of flexible material to extend therethrough, in a position about perpendicular to the longitudinal axis 12a of the elongated tubular member 12. The slots can include a transversal channel with two lateral slots, grooves or wedges, each of the lateral slots, grooves or wedges being disposed on each end of the transversal slot, to allow increased suture positioning for future suture manipulation with the instrument. Openings 73, 74 with transversal suture loading slots form and are part of a securing mechanism 99 provided at the distal end of instrument 100 that is configured to releasably support a plurality of lengths of flexible strands 80, 90 therein. Flexible strands 80, 90 can extend through the openings 73, 74 in similar or different directions and/or orientations and/or locations. Flexible strands 80, 90 can be formed essentially of suture, for example, braided suture that can include fibers of ultrahigh molecular weight polyethylene (UHMWPE). In an embodiment, flexible strands 80, 90 can be formed of elastic suture. In an embodiment, flexible strands 80, 90 can be formed of UHMWPE braided with polyester.

Handle assembly 20 (grip assembly 20) of instrument 100 (illustrated in FIG. 1) is provided at the proximal end 11 of the instrument and includes a pair of cooperating front and rear handles 21, 23 (proximal and distal handles 21, 23), respectively, at the proximal end 11 of shaft 12. The handles 21, 23 are relatively movable to position and reposition the movable jaws 30, 40 between the open position (shown in FIG. 2) and the close position (shown in FIG. 5). The handles 21, 23 each have a finger loop 21a, 23a to accept, for example, the thumb and middle finger of the user. By squeezing the handles 21, 23 towards each other, an operating rod (not shown) can be repositioned to urge the jaws 30, 40 into the close position of FIG. 2. The closing force on the jaws 30, 40 is determined by the amount of pressure applied through the handles 21, 23. Opening of the jaws 30, 40 can be accomplished by spreading the handles 21, 23 away from each other.

Handle assembly 20 provides for opening and closing of the first and second movable jaws 30, 40 coupled to the tip of the instrument 100. Handle assembly 20 can also include a locking mechanism 25 connected to the operating handles 21, 23 for locking and unlocking the jaw assembly 50 (i.e., for locking and unlocking of the first and second movable jaws 30, 40 as desired by the user). In an embodiment, locking mechanism 25 can be a ratchet mechanism 25 that includes an additional finger loop 25a, to allow the user to lock and/or unlock the jaw assembly 50 at different intermediary positions between an open configuration and a fully close position.

Instrument 100 further includes an actuator (not shown), which is provided within the shaft 12 and at the distal end of the shaft. The actuator is connected to the handles 21, 23 and is designed to move the movable jaws 30, 40 from a first position (for example, open position) to a second position (for example, close position) as each of the handles 21, 23 is moved from a first position to a second position, and to grasp and secure a plurality of flexible strands 80, 90 between the movable jaws 30, 40 and the stationary jaw 10 and within the openings 73, 74.

FIGS. 6 and 7 illustrated additional embodiments of jaw assemblies 150, 250 of instruments 200, 300. For simplicity, only the distal end of the instruments 200, 300 are shown. Jaw assembly 150 of FIG. 6 is about similar to jaw assembly 50 of instrument 100 in that jaw assembly 150 is also provided with a stationary jaw 10 and two movable jaws 30a, 40a that form two openings 73a, 74a (enclosures 73a, 74a; through-openings 73a, 74a; apertures 73a, 74a) when in the close configuration. However, movable jaws 30a, 40a are located on opposite sides of the stationary jaw 10 and of the longitudinal axis 12a of the shaft 12. Openings 73a, 74a are separate, distinct, adjacent, and continuous openings. First and second openings 73a, 74a can have similar or different dimensions and can each hold at least one flexible strand such as suture therein. First and second openings 73a, 74a can have a semi-circular configuration or similar configurations.

Jaw assembly 250 of FIG. 7 is about similar to jaw assembly 50 of instrument 100 in that jaw assembly 250 is also provided with a stationary jaw 10b and a movable jaw 30b that form two openings 73b, 74b (enclosures 73b, 74b; through-openings 73b, 74b; apertures 73b, 74b) when in the close configuration. However, instrument 300 includes only one stationary jaw 10b and only one movable jaw 30b. Projection 63b of the stationary jaw 10 engages corresponding projection 33 of the movable jaw 30 to form two openings 73b, 74b, when the jaw assembly 250 is in the close position. Openings 73b, 74b are separate, distinct, adjacent, and continuous openings. First and second openings 73b, 74b can have similar or different dimensions and can each hold at least one flexible strand such as suture therein. First and second openings 73b, 74b can have an oval or elliptical configuration or similar configurations.

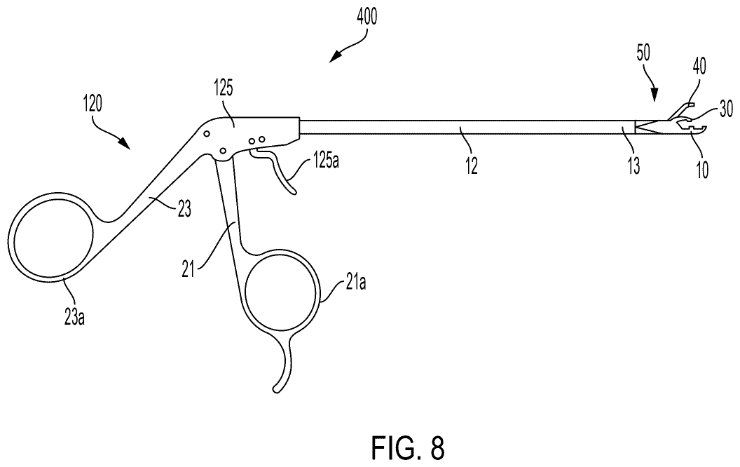

FIG. 8 illustrates another surgical instrument 400 of the present disclosure. Surgical instrument 400 is about similar to surgical instrument 100, described above, in that surgical instrument 400 also includes jaw assembly 50 formed of jaws 10, 30, 40, wherein stationary jaw 10 (stationary member 10; static member 10; tip 10; mounted stationary jaw 10) is provided at the distal end 13 of the instrument and is preferably integral with (fixedly mounted to) the tubular member 12 at the distal end 13 of the shaft; and wherein jaws 30, 40 (first and second movable jaws 30, 40; movable members 30, 40) are movable with respect to the stationary jaw 10 and with respect to each other. However, surgical instrument 400 differs from surgical instrument 100 in the configuration of handle assembly 120.

Handle assembly 120 includes a locking mechanism 125 connected to the operating handles 21, 23 for locking and unlocking the jaw assembly 50, wherein the locking mechanism 125 is a trigger in the form of a lever 125a, to allow the user to lock and/or unlock the jaw assembly 50 at different intermediary positions between an open configuration and a fully close position.

Flexible strand 80, 90 (coupler 80, 90; flexible material 80, 90; suture tape 80, 90; suture 80, 90) can be formed of suture, either round and/or flat suture, for example, a suture strand or suture tape.

A method of manipulating suture with instrument 100, 200, 300 comprises: (i) positioning a first flexible strand 80 and a second flexible strand 90 within a securing mechanism 99 at the tip of instrument 100, 200, 300, 400; and (ii) securing the first flexible strand 80 within a first jaw opening 73, 73a, 73b and securing the second flexible strand 90 within a second jaw opening 74, 74a, 74b.

A method of suturing tissue using the suture passer instrument 100, 200, 300, 400 comprises: (i) loading a first flexible strand 80 onto a first region of a stationary jaw 10 at the tip of instrument 100, 200, 300, 400; (ii) loading a second flexible strand 90 onto a second region of the stationary jaw 10 at the tip of instrument 100, 200, 300, 400; (iii) actuating a handle assembly 20 to advance a first movable jaw 30, 30a towards the stationary jaw 10 from a first position to a second position, to form a first opening or enclosure 73, 73a and to capture the first flexible strand 80 therein; and (iv) actuating the handle assembly 20, 120 to advance a second movable jaw 40, 40a towards the stationary jaw 10 from a first position to a second position, to form a second opening or enclosure 74, 74a and to capture the second flexible strand 90 therein.

A surgical instrument 100, 200, 300, 400 comprises: (i) a shaft 12 having a longitudinal axis 12a, a distal end 13, and a proximal end 11; (ii) a jaw assembly 50, 150, 250 at the distal end 13 of the shaft 12, the jaw assembly 50, 150, 250 being configured to close to form two openings 73, 73a, 73b, 74, 74a, 74b to allow secure placement and holding of different flexible strands 80, 90 (for example, sutures, suture wires, or a similar strands); and (iii) an actuator configured to move the jaw assembly 50, 150, 250. An actuator can be within the shaft 12 and at the distal end of the shaft. A surgical instrument 100, 200, 300, 400 can be a suture grasper, suture retriever, and/or suture passer.

A jaw assembly 50, 150 can include two movable jaws 30, 40, 30a, 40a (movable members 30, 40, 30a, 40a) and a stationary jaw 10 (stationary member 10). Each of the two movable jaws 30, 40, 30a, 40a can be movable with respect to the stationary jaw 10 and to the other movable jaw. The two movable jaws 30, 40, 30a, 40a can have different lengths or can have similar lengths. The two movable jaws 30, 40, 30a, 40a can be located on same side, or on opposite sides of the stationary jaw 10.

A jaw assembly 250 can include one movable jaw 30b (movable member 30b) and a stationary jaw 10b (stationary member 10b). The movable jaw 30b can be movable with respect to the stationary jaw 10b. The movable jaw 30b and the stationary jaw 10b can have about similar lengths and can be located on opposite sides relative to the longitudinal axis 12a of the shaft 12. The movable jaw 30b is actuated to come in contact with the lower jaw 10b and to form two openings or enclosures 73b, 74b for capturing and securely retaining two different flexible strands 80, 90 each within one of the openings.

A suture passing instrument 100, 200, 300, 400 comprises a securing mechanism 99 with two suture securing mechanisms 73, 74. A first suture securing mechanism can include a first continuous opening 73 (enclosure; aperture; through-hole; or slot) formed by a stationary jaw 10 and a first movable jaw 30. Opening 73 houses, captures, retains, secures at least one flexible strand 80 therein. A second suture securing mechanism can include a second continuous opening 74 (enclosure; aperture; through-hole; or slot) formed by a stationary jaw 10 and a second movable jaw 40. Opening 74 houses, captures, retains, secures at least one flexible strand 90 therein.

Flexible strand 80, 90 can be a suture strand or any suture-like material known in the art that could pass through tissue. Flexible strand 80, 90 can be a simple length of a flexible strand or any portion of a flexible strand, for example, a tail of a suture-loop construct, or a middle portion or a terminal portion of a suturing construct (such as a soft suture anchor loaded with a knotless, adjustable loop construct, among many others).

A flexible strand 80, 90 can include a high-strength suture, such as an ultrahigh molecular weight polyethylene (UHMWPE) suture. High strength suture may be a FiberWire® suture sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material.

Flexible strand 80, 90 can be formed of various flexible materials and strands such as round suture, flat suture, ribbon, or flat tape (for example, suture tape) or combination of suture and tape. Exemplary materials can include suture, silk, cotton, nylon, polypropylene, polyethylene, ultrahigh molecular weight polyethylene (UHMWPE), polyethylene terephthalate (PET), and polyesters and copolymers thereof, or combinations thereof. Flexible strand 80, 90 can have cross-sections of various forms and geometries, including round, oval, rectangular, or flat, among others, or combination of such forms and geometries. Flexible strand 80, 90 can be braided or multi-filament suture such as FiberTape® suture tape (as disclosed in U.S. Pat. No. 7,892,256, the disclosure of which is incorporated in its entirety herewith).

Flexible strand 80, 90 can be also formed of a stiff material, or combination of stiff and flexible materials. Various regions, or sections of flexible strand 80, 90 can be coated and/or provided in different colors for easy manipulation during the surgical procedure. Easy identification of suture in situ is advantageous in surgical procedures, particularly during arthroscopic surgeries, endoscopic and laparoscopic procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A surgical instrument comprising:
a shaft having a longitudinal axis, a distal end, and a proximal end;
a jaw assembly located at the distal end and comprising:
a stationary jaw fixedly mounted to the distal end of the shaft, the stationary jaw having two separate contacting sites located on a surface of the stationary jaw; and
a first movable jaw and a second movable jaw, wherein the first movable jaw and the second movable jaw are mounted to the distal end of the shaft; and
a handle assembly configured to move the first movable jaw from a first position to a second position with respect to the stationary jaw and to form a first opening, and to move the second movable jaw from a first position to a second position with respect to the stationary jaw and to form a second opening.

2. The surgical instrument of claim 1, wherein the first opening retains and secures a first flexible strand and wherein the second opening retains and secures a second flexible strand.

3. The surgical instrument of claim 1, wherein at least one of the first flexible strand and the second flexible strand is a suture, suture tape, elastic material, suture tube, or suture sheath.

4. The surgical instrument of claim 1, wherein, when in the second position, a most distal tip of the first movable jaw engages one of the two separate contacting sites of the stationary jaw and forms the first opening.

5. The surgical instrument of claim 1, wherein, when in the second position, a most distal tip of the second movable jaw engages another of the two separate contacting sites of the stationary jaw and forms the second opening.

6. The surgical instrument of claim 1, wherein one of the two contacting sites is a most distal end of the stationary jaw.

7. The surgical instrument of claim 1, wherein the first movable jaw has a first length, and the second movable jaw has a second length.

8. The surgical instrument of claim 7, wherein the first length is different from the second length.

9. The surgical instrument of claim 7, wherein the first length is about equal to the second length.

10. The surgical instrument of claim 1, wherein the handle assembly includes a pair of handles and a locking mechanism.

11. The surgical instrument of claim 10, wherein the locking mechanism includes a ratchet mechanism.

12. The surgical instrument of claim 1, wherein the instrument is a suture grasper, suture retriever, or suture passer.

13. A method of grasping and retaining sutures with a surgical instrument as recited in claim 1 comprising:
positioning a first flexible strand at a first location on the stationary jaw;
positioning a second flexible strand at a second location on the stationary jaw;
moving the first movable jaw from the first position to the second position to capture the first flexible strand within the first opening and at the first location; and
moving the second movable jaw from the first position to the second position to capture the second flexible strand within the second opening and at the second location.

14. The method of claim 13, wherein the steps of moving the first movable jaw and moving the second movable jaw are conducted sequentially.

15. The method of claim 13, wherein the steps of moving the first movable jaw and moving the second movable jaw are conducted simultaneously.

16. The method of claim 13, wherein the second position is a close position.
